(19) United States
(12) Patent Application Publication
(10) Pub. No.: US 2011/0028803 A1
(43) Pub. Date: Feb. 3, 2011

(54) METHOD AND DEVICE FOR NON-INVASIVE DETERMINATION OF THE CONCENTRATION OF A SUBSTANCE IN A BODY FLUID

(76) Inventor: Stig Ollmar, Huddinge (SE)

Correspondence Address: LADAS & PARRY LLP, 224 SOUTH MICHIGAN AVENUE, SUITE 1600, CHICAGO, IL, 60604, US

(21) Appl. No.: 12/935,333

(22) PCT Filed: Mar. 31, 2008
(86) PCT No.: PCT/EP08/53811

§ 371 Date: Sep. 29, 2010

(51) Int. Cl. A61B 5/145

(52) U.S. Cl. 600/301

(57) ABSTRACT

The present invention relates generally to the field of measuring glucose levels in a body fluid of a subject, in particular to non-invasive measuring of blood glucose in blood of a subject. A device is disclosed comprising an electrically conducting probe comprising a plurality of electrodes adapted for measuring electrical impedance of the tissue of a subject, typically skin, a sensing device adapted for sensing at least one concentration of an ion in the tissue, typically acidity (pH), wherein said electrically conducting probe and said sensing device are adapted to obtain said electrical impedance of the tissue and said concentration of an ion in the tissue simultaneously. The device further comprises a blood glucose determining unit adapted for determining an estimate, typically a concentration, of blood glucose based on said electrical impedance of the tissue and said concentration of an ion in the tissue. Further, a device is disclosed comprising an electrically conducting probe comprising a plurality of electrodes adapted for measuring electrical impedance of the tissue of a subject, typically the skin, wherein at least one of said plurality of electrodes is adapted to, when being in voltage mode, sense a signal representative of a concentration of an ion in the tissue, typically acidity (pH), wherein said electrically conducting probe is adapted to simultaneously obtain said impedance of the tissue and said concentration of an ion in the tissue, the device also comprising a blood glucose determining unit adapted for determining an estimate, typically a concentration, of blood glucose based on said electrical impedance of the tissue and said concentration of an ion in the tissue. Furthermore, methods for non-invasive determination of an estimate of blood glucose in blood of a subject are disclosed.

### FIELD OF THE INVENTION

The present invention relates to a method and a device for non-invasive determination of the concentration of a substance in a body fluid of a subject, and in particular to the determination of a blood glucose level of a subject such as a diabetic patient, based on tissue impedance data and concentrations of ions in tissue of the subject, where the tissue typically is skin.

### BACKGROUND OF THE INVENTION

Non-invasive methods for determining glucose concentrations in a body fluid of a subject are generally desirable over invasive methods, that is methods that involve taking samples from the body of a subject. It will be understood that, in particular in the context of the present invention, non-invasive methods means methods where samples of body tissue are not required. Non-invasive techniques are generally more convenient than invasive techniques, for instance, they involve less risk of infection, are less painful, are easier to carry out etc., as will be described in detail in the following.

There are a number of reasons for determining the glucose concentration in a body fluid of a subject. For instance, for a patient suffering from diabetes, it is generally required to frequently monitor the concentration of glucose in the blood of the patient, in general several times daily, in order to know the required amount of insulin that needs to be administered, and at what time. Presently, patients in general rely on self-monitoring of glucose concentrations using an invasive procedure employing a blood glucose meter, which draws a small sample of blood by breaking or lancing the skin of the patient, in order to cause an external flow of blood which is then collected in some way, and then determines the glucose concentration directly from the so collected sample of blood. This method presents several drawbacks. For example, patients may experience discomfort having to take a blood sample repeatedly, several times a day, at regular intervals. Further, a patient may forget to draw blood at a specified time, thereby introducing an error in the glucose monitoring process which is difficult to control, thus lowering the precision in the glucose monitoring. This can lead to that a dose of insulin that is either too small or too large is administered to the diabetic patient, and also possibly at the wrong time. Thus, there is a need within the art for accurate non-invasive methods for determining the glucose concentration in a body fluid of a subject, in particular in the blood of a subject, which alleviates or eliminates the problem associated with the prior art, in particular avoiding the need to draw blood samples, at least on a routine or daily basis, while still maintaining good accuracy in determining glucose concentrations.

There have been several attempts to develop non-invasive techniques for glucose determination that are able to monitor the glucose concentration in blood continuously. Some of these techniques involve measuring electric impedance in body tissue, which is also known as bioimpedance. As known in the art, impedance measurements of body tissue have previously been carried out in order to evaluate or diagnose a number of conditions. The total body tissue impedance depends on a number of factors, such as the composition of cellular structure and intra- and extra-cellular fluids, and is therefore capable of providing useful information for the purpose of determining biological conditions.

Body tissue impedance measurements have been used in a number of applications other than glucose concentration determination, examples of which are: estimation of skin irritation of different chemicals (“Electrical impedance related to experimentally induced changes of human skin and oral mucosa”, I. Nicander, PhD Thesis, Karolinska Institutet, Stockholm (1998)), cardiac monitoring (“Electrical impedance and cardiac monitoring—technology, potential, and application”, M. Min et al., International journal of bioelectromagnetism volume 5, pages 53-56 (2003)), and detection of skin cancer (“Differentiation among basal cell carcinoma, benign lesions, and normal skin using electric impedance”, D. G. Beetner et al., IEEE Trans. Biomedical Eng. volume 50, issue 8, pages 1020-1025 (2003); “Minimally invasive electrical impedance spectroscopy of skin exemplified by skin cancer assessments”, P. Åberg et al., Proc. IEEE EMBS, Cancun (MX), 17-21 Sep. 2003, pages 3212-3214, ISBN 07803-7709-7 (2003)).

Various techniques for determining glucose concentration in a body fluid are known in the art, with or without employing tissue impedance measurements. Techniques that do not employ tissue impedance measurements have for instance been disclosed in U.S. Pat. No. 5,036,861, describing a wrist-mounted device having an electrode which measures the glucose present in the sweat on the skin surface, WO 01/26538, also describing a wrist-mounted device for measuring the glucose level in blood, U.S. Pat. No. 5,222,496, describing an infrared glucose sensor that can be mounted on a wrist, finger, etc., U.S. Pat. No. 5,433,197, describing determination of glucose concentration in the blood of a subject by illuminating the eye of the subject with near-infrared radiation, U.S. Pat. No. 5,115,133, U.S. Pat. No. 5,146,091, and U.S. Pat. No. 5,197,951, describing determination of glucose levels in blood vessels in a tympanic membrane of an ear of a subject by light absorption measurements, and WO 95/04496 and WO 97/39341, describing the use of radio frequency spectroscopy, in vivo or in vitro, in order to determine the concentration of target chemicals in blood, such as glucose.

Furthermore, skin impedance measurements as a tool for determining glucose concentration of a body fluid have for example been disclosed in WO 98/04190 and WO 99/39627, which describe measuring impedance in a body fluid to obtain the glucose concentration, EP 1,437,091, describing a minimally invasive method and apparatus for measuring skin impedance and correlation with the blood glucose concentration, by means of an electrode device having microspikes for penetrating the skin surface, and U.S. Pat. No. 5,353,082, describing a probe having a plurality of electrodes for detecting surface phenomena in body tissue, by measuring impedance of the surface of the body tissue.

According to the prior art, for example CA 2,318,735, describing a method and an apparatus for non-invasively determining the glucose level in a fluid of a subject by measuring skin tissue impedance, and U.S. Pat. No. 6,517,482, describing a method and an apparatus for non-invasively measuring glucose levels in a fluid of a subject, it has proved possible to observe a correlation between the concentration of glucose in blood and electrical impedance parameters of the skin, in some test subjects for a limited amount of time. However, for some test subjects no correlation at all was found, indicating that yet to be discovered parameters were influencing the measurements. One study by Birgersson and Neiderud (“Bioelectrical parameters related to glucose level: measurement principles and data analysis”, U. Birgersson and F. Neiderud, MSc. Thesis, Royal Institute of Technology, Stockholm (2004)) demonstrated a correlation between glucose concentration and skin impedance data both non-invasively and subcutaneously.

Hence, there is a need within the art for means for non-invasive determination of the concentration of a substance such as blood glucose in the blood of a subject, that are quick, easy to carry out, and eliminate or alleviate the disadvantages with the prior art, that generally requires samples of blood to be taken, and at the same time providing comparable or better accuracy as compared to invasive procedures and devices according to the prior art.

### SUMMARY OF THE INVENTION

Thus, it is an object of the present invention to provide a device and a method for determining an accurate and reliable estimate, typically a concentration, of blood glucose in the blood of a subject.

Another object of the present invention is to provide a device and a method for determining an estimate of blood glucose in the blood of a subject that is easy to carry out and that reduces or eliminates the discomfort or inconvenience for the subject.

A further object of the present invention is to provide a device and a method for determining an estimate of blood glucose in the blood of a subject in a swift and efficient manner.

Yet another object of the present invention is to provide a device and a method for determining an estimate of blood glucose in the blood of a subject that enables an early detection of diabetes.

These objects, as well as further objects that will become apparent from the following description and the accompanying claims, are achieved by a device and a method according to the invention.

According to a first aspect of the invention, there is provided a device for non-invasive determination of an estimate of blood glucose, typically glucose concentration, in the blood of a subject, comprising an electrically conducting probe comprising a plurality of electrodes adapted to measure the impedance of the tissue of a subject, typically the skin, and at least one sensor or sensing device adapted to sense at least one concentration of an ion, typically acidity (pH), in the tissue of a subject, wherein the electrically conducting probe and the sensing device are adapted to substantially simultaneously measure the impedance of the tissue and sense a concentration of an ion in the tissue, respectively, and a blood glucose determining unit, adapted to determine an estimate of a blood glucose level of the subject based on the measured tissue impedance and the ion concentration, typically acidity.

In the context of the present invention, by “substantially simultaneously” it is meant, e.g., that the steps of measuring the tissue impedance and sensing at least one value of a concentration of an ion, typically acidity, in the tissue, typically the skin, takes place with only such a short time interval between such that the measurement process is practically feasible, possibly dependent on the particular configuration of the probe. This has the advantage that it ensures that the steps of measuring the tissue impedance and sensing at least one value of a concentration of an ion in the tissue are carried out under quite similar external conditions, for instance essentially the same measurement site of the surface of the tissue, etc., so as not to introduce any artefacts in the so obtained impedance and ion concentration data.

According to a second aspect of the invention, there is provided a device for non-invasive determination of an estimate of blood glucose, typically glucose concentration, in blood of a subject, comprising an electrically conducting probe comprising a plurality of electrodes for measuring the impedance of the tissue of a subject, typically the skin, wherein at least one of the plurality of electrodes is adapted for, when being in voltage mode, to sense a signal representing a value of the concentration of an ion, typically acidity (pH), in the tissue of a subject, and wherein the electrically conducting probe is adapted to substantially simultaneously obtain the value of the impedance of the tissue of a subject and the value of the concentration of an ion in the tissue of a subject. Furthermore, the device according to a second aspect of the invention comprises a blood glucose determining unit, adapted to determine an estimate of blood glucose, typically glucose concentration, of the subject on the basis of the value of the tissue impedance and a value of the concentration of an ion in the tissue.

Thus, the present invention is based on the insight that the acidity (pH) in tissue significantly affects the result when estimating the blood glucose using electrical impedance. Therefore, the devices according to the first and the second aspect of the invention present many advantages, one of which is that they avoid the drawbacks of the prior art, which typically require a blood sample to be taken for determining the concentration of a substance (glucose) in a body fluid (blood), which can present risks or discomforts for the patient. Furthermore, the devices according to the first and second aspects of the invention have an increased accuracy in determining the concentration of a substance, e.g. glucose, compared to the prior art, because the acidity of the tissue can be sensed at the same time as the tissue impedance is obtained, the knowledge of which acidity of the tissue is required to improve the correlation between impedance data and blood glucose, as will be described in detail below. Thus, the devices according to the first and second aspects of the invention are able to show increased correlation between blood glucose and tissue impedance data, as compared to prior art blood glucose determining devices. A further advantage with the devices according to the first and second aspects of the invention is that, in screening for diabetic subjects at health facilities, hospitals, etc., the devices according to the first and second aspects of the invention can provide a much faster screening process as compared to screening employing invasive blood glucose meters, where a sample of blood for each subject is required. Thereby, significant cost savings in the health care can be obtained.

The device according to the second aspect of the invention has the additional advantage, as compared to the device according to the first aspect of the invention, in that it can possibly be manufactured such that it has a smaller size, because it does not require a separate sensing device or sensor for sensing ion concentrations in the tissue, typically acidity (pH), thus being optimized for portable solutions, as well as possibly being cheaper to manufacture.

In the following, various operations will be described as a multiple of discrete steps that are performed in turn in a manner helpful for understanding the invention. However, the order of description should not be construed as to imply that these steps are necessarily performed in the order in which they are presented, or even dependent on the order in which they are presented. Also, “one embodiment” does not necessarily refer to the same embodiment, although it may Furthermore, “an embodiment of the invention” may refer to different aspects of the invention, unless otherwise specified.

According to a third aspect of the invention, there is provided a method for non-invasive determination of an estimate, typically the concentration, of a substance (blood glucose) in a body fluid (blood) of a subject, where the method comprises the steps of: placing an electrically conducting probe against a tissue surface of a subject, typically a surface of the skin, wherein the probe comprises a plurality of electrodes for measuring the impedance of the tissue of a subject and at least one sensing device or sensor, adapted for sensing at least one concentration of an ion, typically acidity (pH), in the tissue of the subject, passing an electrical current through the electrodes to obtain a value of the impedance of the tissue, using the sensing device or sensor to obtain at least one value of a concentration of an ion in the tissue, wherein the value of the impedance of the tissue and the value of a concentration of an ion in the tissue are obtained simultaneously or almost simultaneously, and on the basis of the so obtained impedance value and the at least one value of a concentration of an ion determine an estimate, typically a concentration, of a substance (blood glucose) in the body fluid (blood).

According to a fourth aspect of the invention, there is provided a method for non-invasive determination of an estimate, typically the concentration, of a substance (blood glucose) in a body fluid (blood) of a subject, where the method comprises the steps of: placing an electrically conducting probe against a tissue surface of a subject, typically a surface of the skin, wherein the probe comprises a plurality of electrodes for measuring the impedance of the tissue of a subject, and wherein at least one of the plurality of electrodes is adapted for, when the electrode is in voltage mode, sensing a signal representing a value of a concentration of an ion, typically acidity (pH), in the tissue of the subject, and furthermore passing an electrical current through the electrodes to obtain a value of the impedance of the tissue, using the at least one electrode adapted for sensing a signal representing a value of a concentration of an ion in the tissue of a subject to obtain at least one value of a concentration of an ion in the tissue, wherein the value of the impedance of the tissue and the value of a concentration of an ion in the tissue are obtained simultaneously or almost simultaneously, and on the basis of the so obtained impedance value and the at least one value of a concentration of an ion determine an estimate, typically the concentration, of a substance (blood glucose) in the body fluid (blood).

It is contemplated that the methods according to the third and fourth aspects of the invention can be applied both to human subjects and to subjects of other animals.

An advantage with the methods according to the third and fourth aspects of the invention is that, in screening for diabetic subjects at health facilities, hospitals, etc., the methods according to the first and second aspects of the invention can provide a much faster screening process as compared to screening using methods employing invasive blood glucose meters, where a sample of blood for each subject is required.

According to a fifth aspect of the invention, there is provided an ion-sensitive field effect transistor (ISFET) adapted to be used in conjunction with a device according to a first aspect of the invention.

According to a sixth aspect of the invention, there is provided an ion-sensitive field effect transistor (ISFET) adapted to be used in conjunction with a device according to a second aspect of the invention.

According to embodiments of the invention, respectively, they further comprise soaking the tissue surface in a saline solution or an electrically conductive gel prior to the step of measuring tissue impedance, in order to enhance the conductive contact of the electrodes with the tissue surface during the measuring step.

According to embodiments of the invention, the electrically conducting probe comprises one pair of electrodes, wherein one electrode is a current injection electrode and the other is a voltage sensing electrode. According to further embodiments of the first and second aspects of the invention, the electrically conducting probe comprises two pairs of electrodes, each pair being a current injection electrode and a voltage sensing electrode. This has the advantage of minimizing eventual impedance measurement contact artifacts, by separating current and voltage electrodes. The impedance system can be a two, three, or four pole system.

According to embodiments of the invention, the plurality of electrodes are arranged in a matrix or array adapted for placement on a tissue surface of a subject, typically a surface of the skin, wherein the device according to the first aspect of the invention further comprises a processing unit, adapted for generating electrical impedance tomography images or spectra in the impedance domain, or the plurality of electrodes and the at least one sensor are arranged in a matrix or an array adapted for placement on a tissue surface of a subject, wherein the device according to the first aspect of the invention further comprises a processing unit, adapted for generating electrical impedance tomography images or spectra in the impedance and ion concentration domain. It is contemplated that the so obtained images or spectra can be related to the tissue structure underlying the tissue surface of a subject, e.g. to provide information about structure and composition of tissue and changes in such tissue, e.g. tumours. The processing unit mentioned above preferably is integrated with the blood glucose determining unit.

It can be expected that such diagnosing of tumours, using the devices and/or methods according to the present invention, thus taking into account acidity (pH) of the tissue, is considerably more accurate than diagnosing by means of prior art cancer detection devices and methods using impedance measurements. Note that cancerous tissue has a different metabolism than healthy tissue, and thus has a different acidity level as compared to healthy tissue.

According to embodiments of the invention, the plurality of electrodes are arranged in a matrix or array adapted for placement on a tissue surface of a subject, wherein the device according to the second aspect of the invention further comprises a processing unit, adapted for generating electrical impedance tomography images or spectra in the impedance domain, or in the ion concentration domain, or in the impedance and ion concentration domain. It is contemplated that the so obtained images or spectra can be related to the tissue structure underlying the tissue surface of a subject, e.g. to provide information about structure and composition of tissue and changes in such tissue, e.g. tumours. The processing unit mentioned above preferably is integrated with the blood glucose determining unit. Because, e.g., acidity varies more in the skin than inside the body, it is especially important to take acidity into consideration when looking for tumours in the skin using impedance measurements.

According to embodiments of the invention, the blood glucose determining unit is adapted to send a signal, e.g. an alert, if the blood glucose estimate, obtained by means of the device according to the first or second aspect of the invention, respectively, is below or above predetermined reference glucose levels. Such reference glucose levels may consist of patient-specific glucose reference data obtained by, for instance, follow-up glucose measurements, using non-invasive or even invasive techniques. It is contemplated that such follow-up measurements or the like, for the purpose of establishing reference glucose levels, are required a substantially less number of times compared to the number of blood glucose determinations by means of the device according to the invention, in its intended use. It is to be understood that the signal sent by the blood glucose determining unit can be adapted such to be able to be received by a receiver or station in a number of different communication networks, however possibly not simultaneously. For instance, it is contemplated that the signal from the blood glucose determining unit can be received as a text message in a mobile phone, as an email or some other easily noticable message on a practitioner's laptop or stationary computer, or as an alert in another suitable communications device, as will be apparent to a person skilled in the arts. Such alert signals, sent from the blood glucose determining unit if the blood glucose estimate is below or above predetermined reference glucose levels, would be very advantageous in reminding a patient, e.g., to administer insulin if the blood glucose level is too high. As well known, prolonged periods of high blood glucose levels can lead to a number of serious complications.

According to embodiments of the invention, the device further comprises a communication unit, which is adapted to communicate with at least one external device via at least one communication network. Such an arrangement would allow, for instance, a practitioner to communicate with a blood glucose determining device located on or within a patient, for measuring and/or monitoring blood glucose levels in the patient without the patient having to visit the practitioner. In some embodiments, this is advantageously combined with the blood glucose determining unit being adapted to send a signal, e.g. an alert, if the blood glucose estimate is below or above predetermined reference glucose levels, as described above. Furthermore, such an arrangement would allow communication, for instance wireless communication in a wireless personal area network, such as bluetooth, between the device according to the invention and a laptop, mobile phone, etc. for displaying the blood glucose estimate to the patient and/or alerting the patient. Of course, other communication networks conceivable to a person skilled in the arts are possible.

According to embodiments of the invention, the device further comprises an insulin delivery device, which is adapted to deliver an insulin dose on the basis of a blood glucose estimate, as obtained by means of the device according to the first or second aspect of the invention, respectively. Furthermore, the blood glucose determining unit is adapted to send a blood glucose estimate signal to the insulin delivery device, in response to which signal the insulin delivery device initiates the insulin dose administration to the patient. The insulin delivery device could, for instance, be an insulin pump or the like. Such an insulin delivery device is an advantageous alternative to multiple daily injections of insulin by means of insulin syringes or pens, and allows for intensive insulinotherapy. In some embodiments, the feature of the insulin delivery device is advantageously combined with the blood glucose determining unit being adapted to send a signal, e.g. an alert, if the blood glucose estimate is below or above predetermined reference glucose levels, as described above, and/or with the device according to invention, respectively, further comprising a communication unit, also as described above.

According to embodiments of the invention, the device is adapted to be kept in continual contact with the tissue of the subject, and is optionally adapted for periodic determination of an estimate of blood glucose. According to further embodiments, the features of the immediately foregoing embodiment is combined with the blood glucose determining unit being adapted to send a signal, e.g. an alert, if the blood glucose estimate is below or above predetermined reference glucose levels, as described above, and/or with the device according to the first or second aspect of the invention, respectively, further comprising a communication unit, also as described above, and/or with the device further comprising an insulin delivery device, also as described above. Accordingly, the device could be designed to be, for instance, wrist-, arm-, or ankle-mounted, and periodic or occasional determinations of an estimate of blood glucose could be obtained and further communicated via a communication network to an external device carried by the patient or a practitioner, and/or possibly alerting the patient and/or the practitioner in case hypoglycemia occurs. The device can be mounted on a wrist, arm, ankle, etc., by means of fastening means, such as straps made of a hook-and-loop (velcro) material, belts, bracelets, etc.

According to embodiments of the invention, the device further comprises an internal energy source or means adapted for energy transfer by electromagnetic induction, for instance radio-frequency induction. The internal energy source can for instance be a battery or the like. Such an arrangement would allow for a completely implanted device for non-invasively determining an estimate of blood glucose, with minimal intrusion on the way of life of the patient. The device is preferably implanted in the subcutaneous tissue. Conveniently, the device is placed in the fatty region of the abdominal region, where diabetics generally inject insulin, and there is ample space, in the region of the buttocks, or just below the collarbone, where if required, a pacemaker usually is implanted. According to further embodiments, the feature of the immediately foregoing embodiments is combined with the blood glucose determining unit being adapted to send a signal, e.g. an alert, if the blood glucose estimate is below or above predetermined reference glucose levels, as described above, and/or with the device according to the first or second aspect of the invention, respectively, further comprising a communication unit, also as described above, and/or with the device further comprising an insulin delivery device, also as described above.

According to yet another embodiment of the present invention, the sensor comprises an ion-selective electrode, wherein the ion-selective electrode is a flat pH glass electrode such as known in the art and commercially available, which has good selectivity and sensitivity for singly-charged ions such as H₃O⁺, H⁺, Na⁺, and Ag⁺. Such an electrode is particularly useful for measuring the concentration of hydronium, H₃O⁺, in an aqueous solution, from which the pH value is determined (because the pH value is a measure of the number of protons, which react with water to form hydronium).

According to yet another embodiment of the present invention, the sensor comprises an ion-sensitive field effect transistor (ISFET). With an ISFET it is meant a device known in the art for measuring ion concentrations in a solution, typically pH. When the ion concentration changes, the current flowing through the transistor will change accordingly. An advantage with using ISFETs is that they are small, rugged, and reliable, and can be integrated with the electrode system used for measuring impedance, thus providing a probe having small dimensions convenient for mobile applications, for instance allowing a user to bring the probe to a patient and apply it on the spot. U.S. Pat. No. 6,863,792 discloses an example of such an ISFET.

According to one embodiment of the present invention, each electrode of the plurality of electrodes comprises at least one spike or needle. With a spike or a needle it is here meant a solid microstructure or an elongate microstructure comprising at least one through-going hole, respectively, wherever it occurs in the specification, unless otherwise specified. Such needles or spikes could advantageously be employed for administering insulin to the patient, for instance by arranging a fluid connection between the base of the electrodes on which the spikes or needles are located and an insulin dispenser or container.

According to yet other embodiments of the present invention, each electrode of the plurality of electrodes comprises at least one spike or needle, having a sufficient length to penetrate at least one layer of the skin of a subject, or having a sufficient length to penetrate below the surface of the skin of a subject to the Stratum Germinativum.

According to yet other embodiments of the present invention, each electrode of the plurality of electrodes comprises at least one spike or needle, having a length of at least 20 μm, or at least 30 μm, or at least 40 μm, or at least 50 μm, or at least 60 μm, or at least 70 μm, or at least 80 μm, or at least 90 μm.

According to yet other embodiments of the present invention, each electrode of the plurality of electrodes comprises at least one spike or needle, having a length of up to 250 μm, or up to 240 μm, or up to 230 μm, or up to 220 μm, or up to 210 μm, or up to 200 μm, or up to 190 μm, or up to 180 μm, or up to 170 μm, or up to 160 μm, or up to 150 μm, or up to 140 μm, or up to 130 μm, or up to 120 μm, or up to 110 μm, or up to 100 μm.

According to an embodiment of the present invention, the probe comprises three electrodes, wherein the spikes or needles of the first and second electrodes are laterally spaced apart a first distance from each other, and the spikes or needles of the first and third electrodes are spaced laterally apart a second distance from each other, and the step of passing an electrical current through the electrodes to obtain a value of the impedance of the tissue comprises separately passing an electrical current between the first and second electrodes and between the first and third electrodes to obtain a first and a second impedance value of the tissue. In a further embodiment of the present invention, the first and second distances are different from each other.

According to further embodiments of the present invention, the probe comprises three electrodes according to the embodiment described above, and furthermore, the first distance is between about 0.1 mm and 40 mm, or between about 0.1 mm and 30 mm, or between about 0.1 mm and 25 mm, or between about 0.1 mm and 20 mm, or between about 0.1 mm and 15 mm, or between about 0.2 mm and 10 mm, or between about 0.2 mm and 8 mm, or between about 0.2 mm and 5 mm, or between about 0.2 mm and 3 mm, or between about 0.2 mm and 1.5 mm, or between about 0.2 mm and 1 mm, or between about 0.2 mm and 0.5 mm, or the second distance is between about 1 mm and 50 mm, or between about 1 mm and 40 mm, or between about 1 mm and 30 mm, or between about 1 mm and 25 mm, or between about 1 mm and 20 mm, or between about 1 mm and 15 mm, or between about 1 mm and 10 mm, or between about 1 mm and 9 mm, or between about 1 mm and 8 mm, or between about 1 mm and 7 mm, or between about 2 mm and 8 mm, or between about 3 mm and 7 mm, or between about 4 mm and 7 mm, or between about 4 mm and 6 mm, or is about 5 mm.

According to further embodiments of the present invention, each electrode of the plurality of electrodes comprises at least two spikes or needles, or at least three spikes or needles, or at least four spikes or needles, or at least five spikes or needles, or at least six spikes or needles, or at least seven spikes or needles, or at least eight spikes or needles, or at least nine spikes or needles, or at least ten spikes or needles, or at least twelve spikes or needles, or at least fifteen spikes or needles, or at least eighteen spikes or needles, or at least twenty spikes or needles, or at least twenty-two spikes or needles, or at least thirty spikes or needles, or at least forty spikes or needles, or at least fifty spikes or needles.

According to other embodiments of the invention, the electric current that is passed through the electrodes is an alternate current.

According to yet other embodiments of the invention, the electric current used for measuring the impedance is an alternate current having frequencies between about 10 Hz and about 10 MHz. For example, a number of frequencies can be used to create an impedance spectrum, e.g. a plurality of logarithmically distributed frequencies are used. In alternative embodiment, frequencies between abobut 40 Hz to about 4 MHz are used, for example, a plurality of logarithmically distributed frequencies can be used. In a further embodiment, the frequencies have a range from about 1 kHz and about 1 MHz.

According to further embodiments of the second aspect of the invention, the plurality of electrodes is made of ion-sensitive materials or, in particular, acidity-sensitive materials having a good selectivity for acidity. According to a further embodiment of the second aspect of the invention, at least one of said plurality of electrodes is made of iridium, antimony, palladium, ruthenium, bismuth, or zirconium, or oxides of iridium, antimony, palladium, ruthenium, bismuth, or zirconium.

Such probes can be engineered to increase the accuracy in sensing a concentration of a particular ion, by choosing an ion-sensitive material according to the above in accordance with the ion of interest in the particular case, such that the probe has a good selectivity and sensitivity for the ion of interest. U.S. Pat. No. 6,863,792 discloses an example of an electrochemical detector based on iridium oxide. Also, in a further embodiment of the second aspect of the invention, at least one of the plurality of electrodes is made of compositions of iridium, antimony, palladium, ruthenium, bismuth, or zirkonium, or oxides of iridium, antimony, palladium, ruthenium, bismuth, or zirkonium.

According to embodiments of the first or second aspects of the invention, the device according to the first or second aspect of the invention, respectively, is adapted to pass an electrical current through at least one of the plurality of electrodes, wherein the electrical current has a frequency between about 10 Hz and 10 MHz

### BRIEF DESCRIPTION OF THE DRAWINGS

It should be understood that the exemplary embodiments of the present invention as shown in the figures are for illustrative purposes only. Further embodiments of the present invention will be made apparent when the figures are considered in conjunction with the following detailed description and the appended claims.

FIG. 1 is a schematic view of a spiked electrode probe according to one exemplary embodiment of the present invention.

FIG. 2 is a schematic close-up view of a spiked electrode probe according to one exemplary embodiment of the present invention.

FIG. 3 is a schematic view of the surface of an electrode probe according to another exemplary embodiment of the present invention.

FIG. 4a is a schematic view of the surface of an electrode probe according to yet another exemplary embodiment of the present invention, comprising a flat pH glass electrode.

FIG. 4b is a schematic view of the surface of an electrode probe according to yet another exemplary embodiment of the present invention, comprising a plurality of ion-sensitive field effect transistors.

FIG. 4c is a schematic view of the surface of an electrode probe according to yet another exemplary embodiment of the present invention.

FIG. 5a is a schematic view of a further exemplary embodiment of the probe according to the first or second aspect of the invention.

FIG. 5b is a schematic view of a further exemplary embodiment of the probe according to the first or second aspect of the invention.

FIG. 6a is a schematic view of an exemplary embodiment of the present invention, comprising an insulin delivery device.

FIG. 6b is a schematic view of an exemplary embodiment of the present invention, comprising an implanted insulin delivery device.

FIG. 7 is a schematic view of an exemplary embodiment of the present invention, wherein the device according to the first or second aspect of the invention is a constituent of a wrist-mounted device for continual contact with the tissue of the wearer.

FIG. 8 is a schematic view of one exemplary embodiment of the first or second aspect of the invention, wherein the device according to the first or second aspect of the invention further comprises a communication unit adapted to communicate with an external device via a communication network.

FIG. 9 is a schematic illustration of an exemplary embodiment of the first or second aspect of the invention, wherein the device according to the first or second aspect of the invention further comprises a processing unit for generating electrical impedance tomography images or spectra.

FIG. 10 is a schematic view of an exemplary embodiment of the present invention, comprising means adapted for energy transfer by electromagnetic induction.

### DETAILED DESCRIPTION OF PREFERRED EMBODIMENTS

Preliminary impedance measurements using a precision impedance spectrometer in physiological saline were carried out in a four-pole test chamber made of an acrylic polymer, as a function of frequency and glucose concentration. The glucose concentration in the physiological saline was varied from zero to about 1600 mg/dl in seven steps, and the impedance was measured at frequencies from 40 Hz to 4 MHz in six order-of-magnitude steps, cf. table 1. As shown in table 1, the measured impedance showed no visible dispersion from neither glucose nor acidity in the bio-compatible frequency range. However, as can be seen in table 1, a large shift in acidity, from 6.1 to 2.8, due to addition of hydrogen chloride, more than resets the impedance change due to glucose.

Thereafter, the influence of acidity in tissue was investigated, which revealed that acidity is a strong modulator of the glucose-impedance relationship, as is explained in detail below. Because a glucose response was observed using non-invasive electrodes on skin, that is the stratum corneum, in the study by Birgersson and Neiderud (“Bioelectrical parameters related to glucose level: measurement principles and data analysis”, U. Birgersson and F. Neiderud, MSc. Thesis, Royal Institute of Technology, Stockholm (2004)), it seems that the cellular structure on which impedance measurements are carried out does not have to be alive to elicit a glucose response. Thus, the stratum corneum, though highly insulating and consisting of dead and keratinized flattened cells, still provide a glucose response, according to the study by Birgersson and Neiderud.

Hence, the invention is illustrated below by laboratory feasibility tests on a tissue model to establish that in order to improve correlation between tissue impedance data of a subject and the glucose concentration in the blood of the subject, simultaneous or almost simultaneous measurements of the acidity (pH) of the tissue of the subject are mandatory. This seems to be true for living tissue as well as for dead tissue, such as the stratum corneum of the skin. Other abundant ions in the skin, e.g. ionized sodium, potassium, chlorine, and calcium, as well as amino acids, lactic acid, urocanic acid, ketones, etc. are considered less important than pH, despite all acids being potential proton donors.

Measurements of impedance using a precision impedance spectrometer were carried out in a controlled tissue model, consisting of a yeast structure, intended to be a model structure of living, homogeneous, cellular tissue. For this reason, a base solution was prepared, consisting of 3 kg of yeast, of the brand “Bl∪ kronjäst” commercially available in Sweden (no sugar is added to this product), and 400 ml physiological saline solution, by dissolving the yeast in the saline solution. The acidity of the base solution was between about 4.10 and 4.15, as measured by accurate acidity measuring means as known in the art. Adding NaOH to the solution varied the acidity of the base solution, such that the acidity in one case was between about 5.3 and 5.5, and in another case was between about 6.8 and 7.8. During the measurements a drift in acidity was observed, that was larger the higher the acidity, which indicates that the acidity in the yeast solution adjusts towards the original level of acidity of the base solution. Dissolving glucose in the base solution varied the glucose concentration in the tissue model.

The normal acidity range for human blood is about 7.38-7.42. The acidity (pH) of blood from a subject suffering from light diabetic acidosis is in the range of about 7.2-7.3, whereas the acidity of blood from a subject suffering from moderate and severe diabetic acidosis is in the range of about 7.0-7.2 and less than 7.0, respectively. For a non-diabetic subject, the intracellular acidity is about 5, and the extra cellular acidity is like blood, according to the above. For a non-diabetic subject, the acidity in skin is about 6. For subjects suffering from diabetes, this value is lower.

Various impedance parameters for the yeast solution were measured using a precision impedance spectrometer as a function of frequency, at different acidity levels and glucose concentrations, as will be described in detail in the following.

First, the total impedance, the phase, and real and imaginary parts of the impedance of the tissue model were measured as a function of frequency, for different levels of acidity (pH) and glucose concentrations, the results of which are shown in the accompanying table 2.

In the tissue model, there are relative changes in impedance parameters due to higher glucose concentration almost at the level of what have been observed earlier in vivo. In view of the good correlation between skin impedance measurements and glucose observed during certain conditions, see for instance CA 2,318,735 and U.S. Pat. No. 6,517,482, the fact that for some test subjects a correlation was observed in many measurements during several days, while for other test subjects correlation was observed at the beginning but diminished or vanished in a few hours, and the fact that a glucose response was elicited both non-invasively and subcutaneously, it is indicated that there are yet to be discovered factors influencing the impedance measurements, one likely dominant candidate of which is the acidity of the tissue, for example the skin. Another possible factor is the temperature, which is generally at a stable level inside the body of a subject, but can show more variation in the skin. However, though not wishing to be bound by any particular theory, it is the applicants' belief that acidity is the dominating factor.

As evident from table 2, there is a dispersion at around 1 MHz, about one order of magnitude higher than in firm biological tissue, but about half the characteristic frequency of, e.g., blood. These results also indicate that the glucose concentration-impedance relationship is non-linear.

Table 3 presents parts of the data presented in table 2.

Tables 4 and 5 show the long indices L-MIX, L-PIX, L-RIX, and L-IMIX, which are impedance parameters according to the definition below, and variation of the long indices, as a function of acidity and glucose concentration. The long indices shown in tables 4 and 5 were determined as follows, similarly to, e.g., the teachings of CA 2,318,735 and U.S. Pat. No. 6,517,482:

L-MIX (magnitude index)=abs(Z_{20 kHz})/abs(Z_{5 MHz}),

L-PIX (phase index)=arg(Z_{20 kHz})/arg(Z_{5 MHz}),

L-RIX (real part index)=Re(Z_{20 kHz})/abs(Z_{5 MHz}),

L-IMIX (imaginary part index)=Im(Z_{20 kHz})/abs(Z_{5 MHz}),

where abs(Zᵢ) is the magnitude (modulus) of the complex electrical impedance at the frequency i, arg(Zᵢ) is the argument (phase angle) in degrees, Re(Zᵢ) is the real part of the complex electrical impedance, and Im(Zᵢ) is the imaginary part of the complex electrical impedance. The real and imaginary parts are calculated from the magnitudes and phase angles as follows: Re(Zᵢ)=abs(Zᵢ)cos [arg(Zᵢ)], and Im(Zᵢ)=abs(Zᵢ)sin [arg(Zᵢ)].

The L-RIX index mainly reflects changes in conductivity, the L-IMIX index reflects changes along the length of the vector describing the impedance in complex space, which will be emphasized if the real and imaginary parts change in the same direction and proportion, and the L-PIX index will be emphasized if the real and imaginary parts change in different directions and/or proportions.

Table 6 shows changes in the absolute values of the total impedance, the phase, and real and imaginary parts of the impedance of the tissue model, in percent per 100 mg/ml glucose or in percent per unit of acidity (pH), for different frequencies, different concentrations of glucose, and different values of acidity.

Normalised to physiological ranges, the best glucose response is from the imaginary part of the impedance at low frequencies and the long index L-IMIX. As can be seen from tables 5 and 6, this response is in the range from about 5 to 10 percent per 100 mg/dl glucose. Thus, the yeast tissue model gives a similar glucose response as observed in earlier in vivo experiments for some subjects. Tables 5 and 6 also show an acidity response for selected impedance parameters of roughly the same numbers, that is about 5 to 10 percent, in terms per 0.2 units pH. When taking into account that acidity in human blood varies with about ±0.1 and in skin with about ±0.5, and also that this variation is even larger for diabetics, it can be concluded from the presented results that acidity and glucose concentration are factors of roughly equal weight in modulating the outcome of tissue impedance measurements, and thus, acidity must necessarily be taken into account when trying to improve correlation between glucose concentration and tissue impedance data.

It should be noted that both acidity in the tissue and glucose concentration in the blood of a subject are varying in unpredictable and uncontrollable ways, depending on eating habits, state of health, etc. of the subject. Thus, it is remarkable that such good correlation between tissue impedance data and glucose concentration in blood has been obtained in earlier studies for a number of test subjects, which suggests that the tissue acidity level is more stable in some test subjects than in others. It further seems that the tissue acidity level is less stable in diabetic test subjects.

It should further be noted that it is to be expected that diagnosing of tumours, using the devices and/or methods according to the present invention, and thus taking into account acidity (pH) of the tissue, is more accurate than diagnosing by means of prior art cancer detection devices and methods using impedance measurements. Note that cancerous tissue has a different metabolism than healthy tissue, and thus generally has a different acidity level as compared to healthy tissue.

Preferred embodiments of the present invention will now be described for the purpose of exemplification with reference to the accompanying drawings, wherein like numerals indicate the same elements throughout the views. It should be understood that the present invention encompasses other exemplary embodiments that comprise combinations of features as described in the following. Additionally, other exemplary embodiments of the present invention are defined in the appended claims.

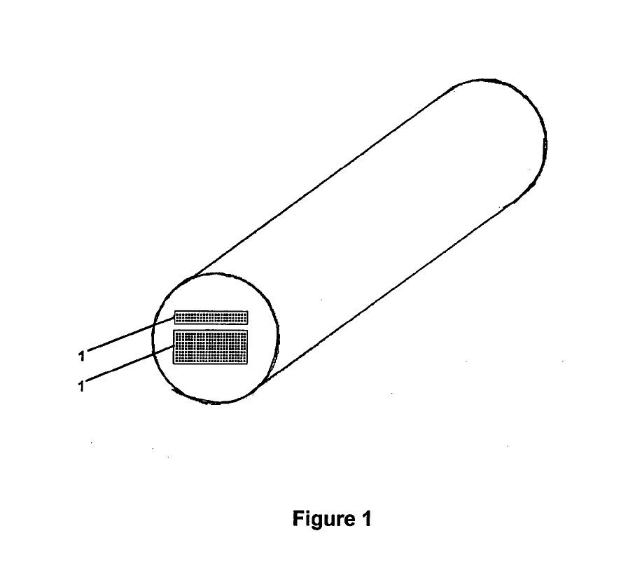

FIG. 1 schematically shows one exemplary embodiment of the first or second aspect of the invention, wherein each of the plurality of electrodes, arranged on panels 1 located at one end of the probe, comprises at least one microstructure in the shape of a spike or needle. In the context of the present invention, by a spike and a needle it is meant a solid microstructure and an elongate microstructure comprising at least one through-going hole, respectively. Such needles or spikes could advantageously be employed for administering insulin to the patient, for instance by arranging a fluid connection between the base of the electrodes on which the spikes or needles are located and an insulin dispenser or container.

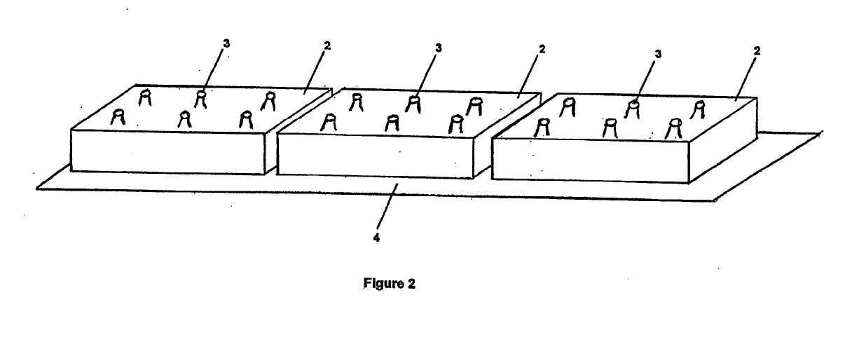

FIG. 2 is a schematic close-up view of one embodiment of the first or second aspect of the invention, wherein the probe comprises three electrodes 2, each having a plurality of spikes 3. The electrodes are supported by a substrate 4.

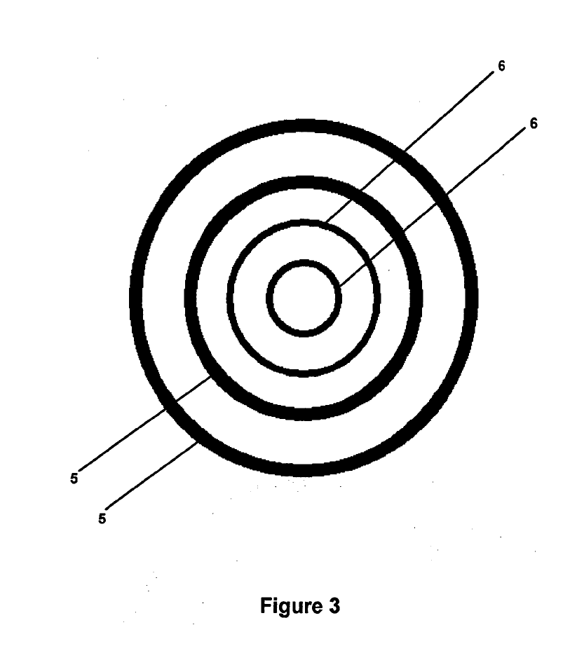

FIG. 3 schematically shows the surface of one exemplary embodiment of the first or second aspect of the invention, wherein the electrically conducting probe comprises two pairs of electrodes in the shape of concentric circles, each pair being a current injection electrode 5 and a voltage sensing electrode 6. Naturally, the exemplary electrodes 5 and 6 as shown in FIG. 3 need not necessarily be arranged in the shape of concentric circles, but may be adapted to adopt any geometric shape according to design or manufacturing requirements.

FIGS. 1-3 do not show the glucose-determining unit of the invention. However, this is not to be construed as if the embodiments according to FIGS. 1-3 are lacking the glucose determining unit. The glucose determining unit is illustrated in exemplary embodiments presented below.

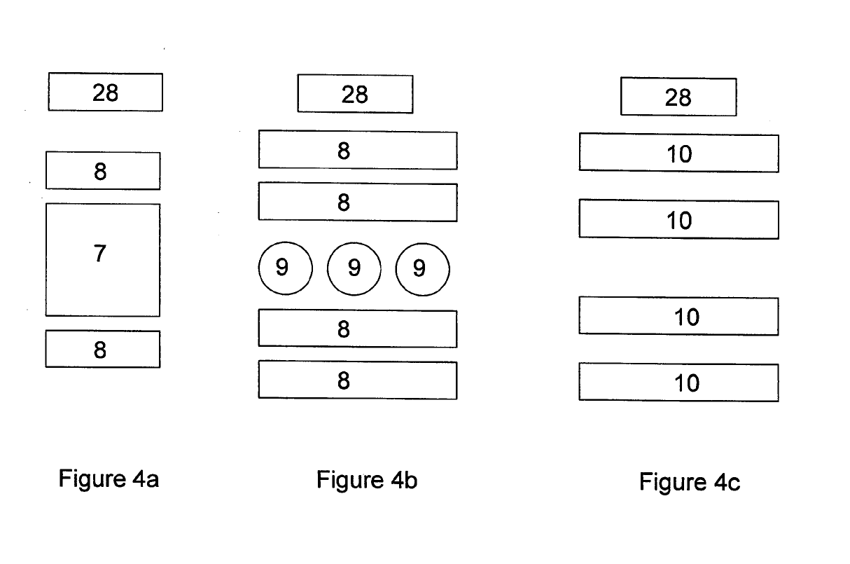

FIGS. 4a, 4b, and 4c show exemplary embodiments of the first and second aspects of the invention.

FIG. 4a schematically shows the surface of an electrically conducting probe according to an exemplary embodiment of the first aspect of the invention, wherein a flat pH glass electrode 7 is arranged on the surface for measuring the concentration of an ion, typically acidity, in the tissue of a subject, typically the skin, and two electrodes 8 can be used for measuring the electrical impedance of the tissue of the subject. Of course, a number of flat glass electrodes 7 could be arranged on the surface, each adapted for sensing the concentration of a particular ion in the tissue, and further electrodes 8 for measuring tissue impedance could be mounted on the surface of the probe, as well. The probe further comprises a glucose determining unit 28, adapted to determine an estimate of blood glucose of a subject based on the tissue impedance and the ion concentration in the tissue.

FIG. 4b schematically shows the surface of an electrically conducting probe according to an exemplary embodiment of the first aspect of the invention, wherein a plurality of ion-sensitive field effect transistors 9, ISFETs, are arranged on the surface for measuring the concentration of an ion, typically acidity (pH), in the tissue of a subject, typically the skin, wherein the ISFETs 9 can be adapted for sensing the concentration of different ions in the tissue. Also, two pairs of electrodes 8, similarly to FIG. 3, are arranged on the surface for measuring tissue impedance. The probe shown in FIG. 4b further comprises a glucose-determining unit 28, adapted to determine an estimate of blood glucose of a subject based on the tissue impedance and the ion concentration in the tissue.

FIG. 4c schematically shows the surface of an electrically conducting probe according to an exemplary embodiment of the second aspect of the invention, wherein at least one of the electrodes 10 is adapted for, when being in voltage mode, sensing a signal representing a value of the concentration of an ion, typically acidity, in the tissue of a subject. This is facilitated by the at least one of the plurality of electrodes 10 being made of an ion-sensitive material, according to one exemplary embodiment of the second aspect of the invention. According to further embodiments of the second aspect of the invention, the at least one electrode is made of an acidity-sensitive material, iridium, antimony, palladium, ruthenium, bismuth, zirconium, etc., or oxides thereof, or composites thereof. Concurrently, the electrodes 10 can be used for tissue impedance measurements, prior to or after obtaining the value of the concentration of an ion, typically acidity. Also, the probe shown in FIG. 4c comprises a glucose determining unit 28, adapted to determine an estimate of blood glucose of a subject based on the tissue impedance and the ion concentration in the tissue.

The glucose determining unit 28 as shown in FIGS. 4a, 4b, and 4c can be integrated with the other components of the device according to the invention, or it can be external, as will be discussed in the following.

It is to be understood that the electrodes and/or sensing devices can be arranged in different configurations, having any geometrical shape, e.g. with the surface of the electrodes and/or sensing devices having the shape of squares, circle sections, ellipses, etc., or having a three-dimensional shape according to a needle, a spike, a bar or rod, etc., and are not limited to the exemplary embodiments as discussed above. It is also to be understood that the electrically conducting probe according to the first or second aspect of the invention may be adapted for placement on the skin of a subject or under the skin of a subject (subcutaneously), for example in the fatty part of the abdominal region, where diabetics generally inject insulin, and there is ample space, or in the proximity of the buttocks. For instance, the probe can be configured as a needle comprising an ISFET 9 and a plurality of concentric electrodes (5, 6) for measuring impedance, or a needle comprising a plurality of electrodes (10) made of acidity-sensitive materials, thus not requiring an ISFET, as discussed elsewhere in the description.

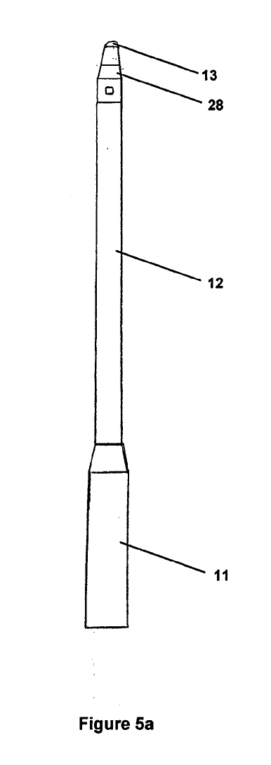

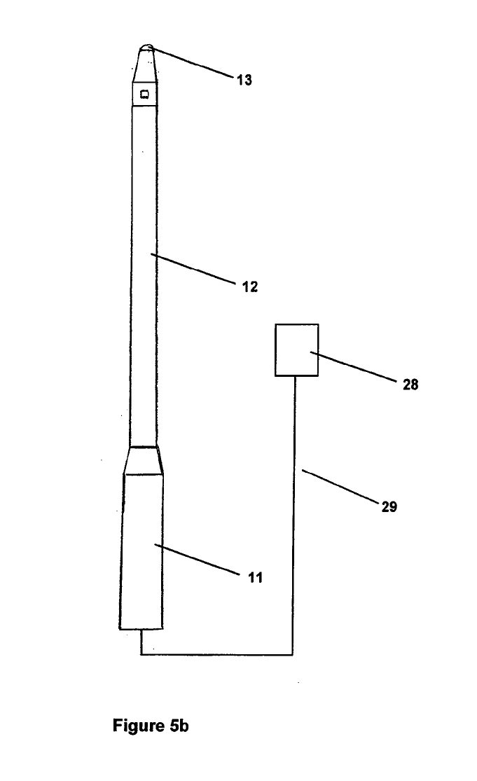

Accordingly, FIG. 5a shows an exemplary embodiment of the probe according to the invention, comprising a handle 11, a rod 12, and a tip 13. The tip 13 comprises a plurality of electrodes for measuring tissue impedance and/or a concentration of an ion, typically pH, in the tissue. Some examples of electrode arrangements have been presented above. Also, the tip 13 either optionally comprises a flat pH glass electrode 7 or an ISFET 9, or at least one of the plurality of electrodes is made of an ion-sensitive material, or an acidity-sensitive material, as discussed previously. The device shown in FIG. 5a further comprises a blood glucose-determining unit 28, preferably integrated with the other components, for instance located immediately below the tip 13 such as shown in FIG. 5. Alternatively, the blood glucose determining unit 28 can be external and connected to the rest of the components by connecting means, for instance connected by a connecting wire 29, as shown in FIG. 5b. A device such as the one shown in FIG. 5a or 5b would be suitable for use by diabetics for self-monitoring of blood glucose, but would also be suitable in screening for diabetic subjects at health facilities, hospitals, etc., or for subcutaneous measurements as discussed above.

It is to be understood that the glucose determining device 28 can be either external or internal, as exemplified above, without this necessarily being mentioned in conjunction with a specific embodiment.

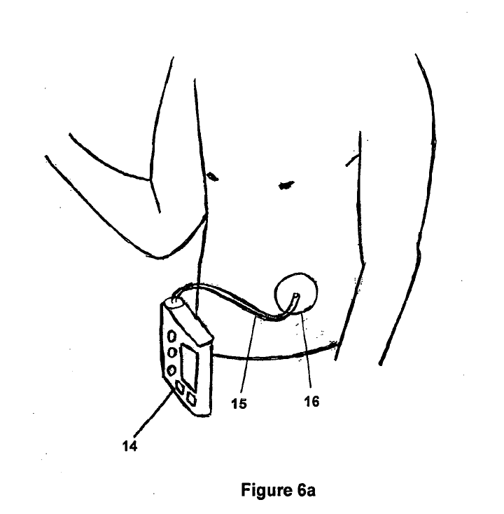

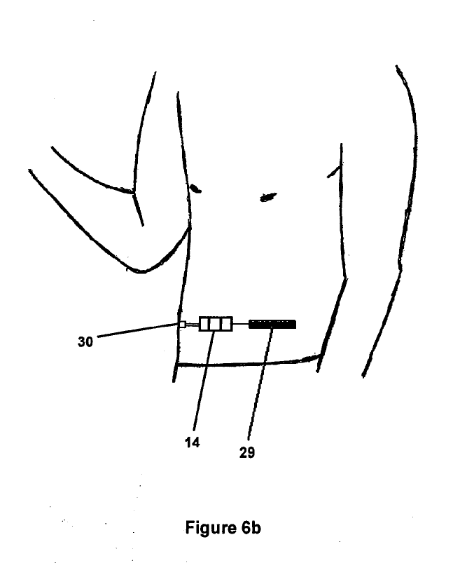

FIG. 6a shows an exemplary embodiment of the invention, where the device according to the first or second aspect of the invention comprises an insulin delivery device, in this particular case exemplified by an insulin pump 14. Insulin is delivered by means of flexible tubing 15 from the pump 14 to the infusion set 16, which typically comprises a cannula (not shown) inserted under the skin for delivery of insulin to the patient. In this example, the device according to the first or second aspect of the invention is located in the infusion set 16, positioned against a skin surface of the patient. The device is adapted to deliver an insulin dose on the basis of a blood glucose estimate, as obtained by means of the device according to the first or second aspect of the invention. Furthermore, the blood glucose determining unit is adapted to send a blood glucose estimate signal to the insulin delivery device (in this case, the insulin pump 14), in response to which signal the insulin delivery device 14 initiates administration of an insulin dose to the patient.

FIG. 6b shows another embodiment of the present invention, where the device according to the first or second aspect of the invention comprises an insulin delivery device, in this particular case exemplified by an insulin pump 14, wherein the insulin pump 14 and the device 29 according to the first or second aspect of the invention are implanted. The device 29 and the insulin pump 14 or other insulin delivery device are preferably connected by a connecting wire, as shown in FIG. 6b, or integrated. In this example, the insulin delivery device can be refilled with insulin through tubing connected to an inlet 30, for instance a catheter, located on the skin of the patient. For the purposes of explaining the invention, the above-mentioned components are visible in FIG. 6b. The device 29 is adapted to deliver an insulin dose on the basis of a blood glucose estimate, as obtained by means of the device 29. Furthermore, the blood glucose determining unit is adapted to send a blood glucose estimate signal to the insulin delivery device (in this case, the insulin pump 14), in response to which signal the insulin delivery device 14 initiates administration of an insulin dose to the patient. In some embodiments, this is advantageously combined with the device 29 further comprising an internal energy source or means adapted for energy transfer, for example, by means of electromagnetic induction, as will be described below.

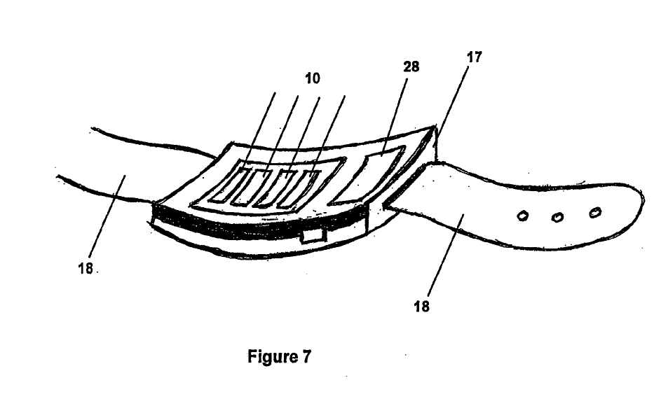

In use, the device according to the first or second aspect of the invention may be designed as a constituent of, for instance, a wrist-mounted device like a wristwatch, as exemplified in FIG. 7, for continuous monitoring of, e.g., glucose, by continual contact with the skin of the wearer, or a mobile telephone having electrically conducting probes according to the invention in a headset, for continual contact with the skin at the outer end of the auditory duct of the wearer. FIG. 7 shows a device 17 according to a second aspect of the invention, having straps 18 for mounting the device on a wrist, ankle, etc. of a patient, an electrode configuration according to the previously disclosed example in FIG. 4c, wherein at least one of the electrodes 10 is adapted for, when being in voltage mode, sensing a signal representing a value of the concentration of an ion, typically acidity, the skin of a subject for the purpose of determining an estimate of blood glucose of the blood of the wearer, as previously described, and a blood glucose determining unit 28. It is to be understood that further applications of the invention, in addition to those above, are conceivable for a person skilled in the arts.

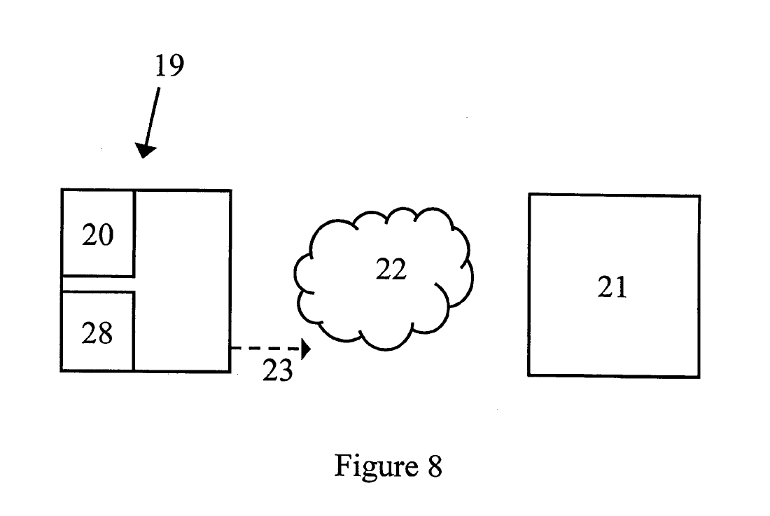

FIG. 8 schematically shows one exemplary embodiment of the first or second aspect of the invention, wherein the device 19 according to the first or second aspect of the invention further comprises a communication unit 20, which is adapted to communicate with at least one external device 21 via at least one communication network 22. The external device 21 could be a mobile phone, a laptop, etc. Such an arrangement would allow, for instance, a practitioner to communicate with a blood glucose determining device located on a patient, for measuring and/or monitoring blood glucose levels in the patient without the patient having to visit the practitioner. In some embodiments, this is advantageously combined with the blood glucose determining unit being adapted to send a signal 23, e.g. an alert, if the blood glucose estimate is below or above predetermined reference glucose levels. Furthermore, such an arrangement would allow communication, for instance wireless communication in a wireless personal area network 22, such as bluetooth, between the device according to the invention and a laptop, mobile phone, etc. for displaying the blood glucose estimate to the patient or a practitioner and/or alerting the patient or a practitioner. Of course, other communication networks 22 conceivable to a person skilled in the arts are possible. Also shown in FIG. 8 is the blood glucose measuring unit 28. The electrodes, and optionally the at least one sensing device, are not shown in FIG. 8.

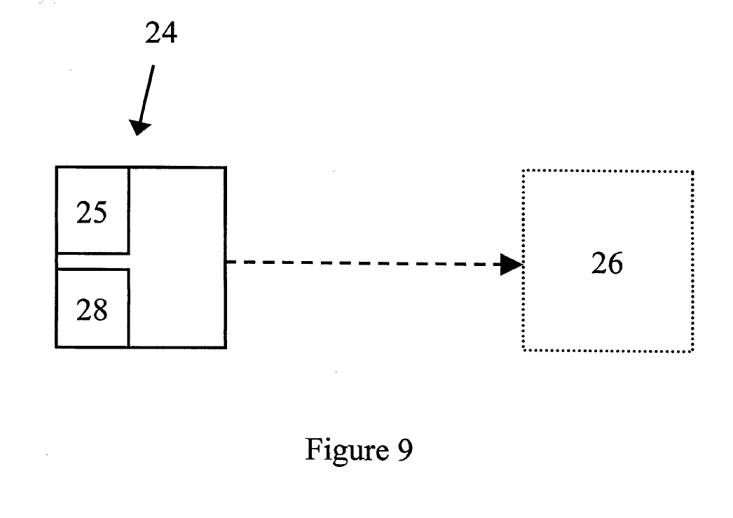

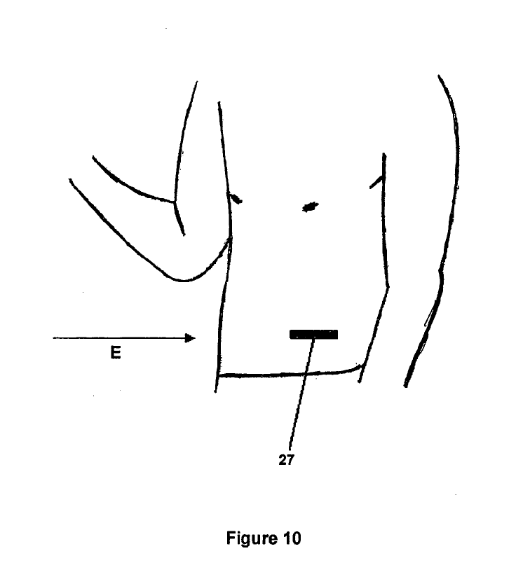

FIG. 9 illustrates one exemplary embodiment of the first aspect of the invention, wherein a device 24 according to the first aspect of the invention further comprises a processing unit 25, adapted for generating electrical impedance tomography images or spectra 26 in the impedance domain, or adapted for generating electrical impedance tomography images or spectra 26 in the impedance and ion concentration domain, and wherein the plurality of electrodes, or the plurality of electrodes and the sensing device, are arranged in matrix or array adapted for placement on a tissue surface of a subject. It is contemplated that such images or spectra 26 can be related to the tissue structure underlying the tissue surface of the subject. Moreover, the processing unit 25 is preferably integrated with the blood glucose determining unit 28. Alternatively, the blood glucose determining unit can be separate from the processing unit 28, as illustrated in FIG. 9.

FIG. 9 further illustrates one exemplary embodiment of the second aspect of the invention, wherein a device 24 according to the second aspect of the invention further comprises a processing unit 25, adapted for generating electrical impedance tomography images 26 in the impedance domain, or adapted for generating electrical impedance tomography images 26 in the impedance and ion concentration domain, and wherein the plurality of electrodes are arranged in matrix or array adapted for placement on a tissue surface of a subject. It is contemplated that such images 26 can be related to the tissue structure underlying the tissue surface of the subject. The processing unit 25 is preferably integrated with the blood glucose determining unit. Alternatively, the blood glucose determining unit can be separate from the processing unit 28, as illustrated in FIG. 9.

FIG. 10 illustrates one exemplary embodiment of the first or second aspect of the invention, wherein the device according to the first or second aspect of the invention 27 comprises means adapted for energy transfer by electromagnetic induction, for instance radio frequency induction, when the device 27 is subjected to an electrical field E, cf. FIG. 10. Such an arrangement would allow for a completely implanted device 27 capable of occasional or periodic non-invasive determination of an estimate of blood glucose, with minimal intrusion on the way of life of the patient. For the purposes of explaining the invention, the implanted device 27 is visible in FIG. 10. The device is preferably implanted in the subcutaneous tissue. Conveniently, the device is placed in the fatty region of the abdominal region, as shown in FIG. 10, where diabetics generally inject insulin, and there is ample space, or in the region of the buttocks, or just below the collarbone, where if required, a pacemaker usually is implanted. For the purpose of implanting the device 27, it can alternatively comprise an internal source of energy, such as a battery. According to a non-limiting example, the device 27 is preferably arranged so that it has the shape of a capsule, about 40 mm long and about 4 mm thick, suitable for being implanted in the body of a patient.

Finally, it is contemplated that the device according to the first or second aspect of the invention comprises the necessary electronics to perform impedance measurements, sensing, and analysis to facilitate determination of an estimate of a substance in a body fluid of a subject, as is known to a person skilled in the arts.

TABLE 1
Impedance measurements in a physiological saline solution carried out in a four-pole
test chamber of an acrylic polymer, as a function of frequency f, and acidity (pH).

| | | | | | | | | pH = 2.8 |
|---|---|---|---|---|---|---|---|---|
| | PH = 5.7 | | | pH = 6.9 | | | pH = 6.1 | (added HCl) |
| | G = 0 | G = 50 | G = 100 | G = 200 | G = 400 | G = 800 | G = 1600 | G = 1600 |
| f (kHz) | mg/dl | mg/dl | mg/dl | mg/dl | mg/dl | mg/dl | mg/dl | mg/dl |
| 0.04 | 166.5 | 165.1 | 164.7 | 164.8 | 165.3 | 166.7 | 170.6 | 164.4 |
| 0.4 | 165.2 | 163.8 | 163.4 | 163.5 | 164.0 | 165.3 | 169.3 | 163.3 |
| 4 | 165.0 | 163.6 | 163.2 | 163.3 | 163.8 | 165.1 | 169.1 | 163.1 |
| 40 | 164.8 | 163.5 | 163.1 | 163.1 | 163.8 | 165.0 | 169.0 | 162.9 |
| 400 | 164.8 | 163.5 | 163.1 | 163.1 | 163.7 | 165.0 | 169.0 | 162.9 |
| 4000 | 180.1 | 178.8 | 178.6 | 178.7 | 178.1 | 179.3 | 183.6 | 177.2 |

The impedance data is given in Ω. The acidity was not altered for glucose concentrations up to 800 mg/dl. At the glucose concentration 1600 mg/dl, the acidity was changed from 6.1 to 2.8 due to addition of HCl.

TABLE 2
Total impedance Z (in Ω), phase φ (in degrees), real and
imaginary parts of Z (in Ω) for a yeast tissue model (see the
text), as a function of frequency f, acidity (pH), and glucose concentration G.

| | f | | | | | | | | | G |
|---|---|---|---|---|---|---|---|---|---|---|
| PH | (MHz) | 0.02 | 0.05 | 0.1 | 0.2 | 0.5 | 1.0 | 2.0 | 5.0 | (mg/dl) |
| 6.8- | Z | 878 | 876 | 874 | 863 | 803 | 671 | 519 | 332 | 400 |
| 7.8 | −φ | 1.0 | 2.3 | 4.4 | 8.7 | 20.4 | 34.4 | 45.4 | 64.9 | |
| | Re Z | 878 | 875 | 871 | 853 | 753 | 554 | 364 | 141 | |
| | Im Z | 15 | 35 | 67 | 131 | 280 | 379 | 370 | 300 | |
| | Z | 1106 | 1102 | 1098 | 1082 | 992 | 805 | 605 | 382 | 800 |
| | −φ | 1.2 | 2.7 | 5.0 | 9.8 | 23.0 | 38.3 | 49.6 | 68.2 | |
| | Re Z | 1106 | 1101 | 1094 | 1066 | 913 | 632 | 392 | 142 | |
| | Im Z | 23 | 52 | 96 | 184 | 388 | 499 | 461 | 355 | |
| | Z | 745 | 743 | 741 | 734 | 690 | 590 | 467 | 313 | nominal |
| | −φ | 0.9 | 2.1 | 3.9 | 7.7 | 18.4 | 31.5 | 42.1 | 61.0 | |
| | Re Z | 745 | 743 | 739 | 727 | 655 | 503 | 347 | 152 | |
| | Im Z | 12 | 27 | 50 | 98 | 218 | 308 | 313 | 274 | |
| 5.3- | Z | 964 | 961 | 957 | 939 | 861 | 700 | 532 | 350 | 400 |
| 5.5 | −φ | 1.1 | 2.6 | 4.9 | 9.5 | 22.4 | 36.9 | 47.3 | 64.8 | |
| | Re Z | 964 | 960 | 954 | 926 | 796 | 560 | 361 | 149 | |
| | Im Z | 19 | 44 | 82 | 155 | 328 | 420 | 391 | 317 | |
| | Z | 1315 | 1311 | 1304 | 1279 | 1141 | 884 | 638 | 367 | 800 |
| | −φ | 1.4 | 3.2 | 5.9 | 11.6 | 26.7 | 42.9 | 53.8 | 71.2 | |
| | Re Z | 1315 | 1309 | 1297 | 1253 | 1019 | 648 | 377 | 118 | |
| | Im Z | 32 | 73 | 134 | 257 | 513 | 602 | 515 | 347 | |
| | Z | 885 | 882 | 879 | 867 | 800 | 657 | 501 | 321 | nominal |
| | −φ | 1.1 | 2.5 | 4.6 | 9.1 | 21.4 | 35.6 | 45.9 | 62.6 | |
| | Re Z | 885 | 881 | 876 | 856 | 745 | 534 | 349 | 148 | |
| | Im Z | 17 | 38 | 70 | 137 | 292 | 382 | 360 | 285 | |
| 4.10- | Z | 1266 | 1261 | 1253 | 1224 | 1068 | 805 | 576 | 339 | 400 |
| 4.15 | −φ | 1.5 | 3.4 | 6.4 | 12.6 | 28.5 | 44.0 | 53.5 | 69.9 | |
| | Re Z | 1266 | 1259 | 1245 | 1195 | 939 | 579 | 343 | 117 | |
| | Im Z | 33 | 75 | 140 | 267 | 510 | 559 | 463 | 318 | |
| | Z | 1606 | 1598 | 1585 | 1542 | 1313 | 952 | 661 | 372 | 800 |
| | −φ | 1.7 | 4.0 | 7.3 | 14.3 | 31.8 | 48.3 | 57.9 | 73.5 | |
| | Re Z | 1605 | 1594 | 1572 | 1494 | 1116 | 633 | 351 | 106 | |
| | Im Z | 48 | 111 | 201 | 381 | 692 | 711 | 560 | 357 | |
| | Z | 1211 | 1206 | 1200 | 1174 | 1032 | 784 | 563 | 338 | nominal |
| | −φ | 1.4 | 3.4 | 6.2 | 12.2 | 27.8 | 43.3 | 52.9 | 68.5 | |
| | Re Z | 1211 | 1204 | 1193 | 1147 | 913 | 571 | 340 | 124 | |
| | Im Z | 30 | 72 | 130 | 248 | 481 | 538 | 449 | 314 | |

TABLE 3
Absolute values of Z (in Ω), φ (in degrees), Re Z (in Ω), and Im Z (in
Ω), respectively, for the yeast tissue model (see the text) at different
frequencies f, as a function of acidity (pH) and glucose concentration G.

| | | |
|---|---|---|
| pH = 4.1 | pH = 5.4 | pH~7.3 |

At f = 20 kHz

| | | | |
|---|---|---|---|
| G = 800 mg/dl | 1606; 1.7; 1605; 48 | 1315; 1.4; 1315; 32 | 1106; 1.2; 1106; 23 |
| G = 400 mg/dl | 1266; 1.5; 1266; 33 | 964; 1.1; 964; 19 | 878; 1.0; 878; 15 |
| G = nominal | 1211; 1.4; 1211; 30 | 885; 1.1; 885; 17 | 745; 0.9; 745; 12 |

At f = 500 kHz

| | | | |
|---|---|---|---|
| G = 800 mg/dl | 1313; 31.8; 1116; 692 | 1141; 26.7; 1019; 513 | 992; 23.0; 913; 388 |
| G = 400 mg/dl | 1068; 28.5; 939; 510 | 861; 22.4; 796; 328 | 803; 20.4; 753; 280 |
| G = nominal | 1032; 27.8; 913; 481 | 800; 21.4; 745; 292 | 690; 18.4; 655; 218 |

At f = 5 MHz

| | | | |
|---|---|---|---|
| G = 800 mg/dl | 372; 73.5; 106; 357 | 367; 71.2; 118; 347 | 382; 68.2; 142; 355 |
| G = 400 mg/dl | 339; 69.9; 117; 318 | 350; 64.8; 149; 317 | 332; 64.9; 141; 300 |
| G = nominal | 338; 68.5; 124; 314 | 321; 62.6; 148; 285 | 313; 61.0; 152; 274 |

TABLE 4
Long indices L-MIX, L-PIX, L-RIX, and L-IMIX, respectively,
for the yeast tissue model (see the text), as a function of
acidity (pH) and glucose concentration G.

| | pH = 4.1 | pH = 5.4 | pH~7.3 |
|---|---|---|---|
| G = 800 mg/dl | 4.32; 71.8; 4.31; | 3.58; 69.8; 3.58; | 2.90; 67.0; 2.90; |
| | 0.129 | 0.0872 | 0.0602 |
| G = 400 mg/dl | 3.73; 68.4; 3.73; | 2.75; 63.7; 2.75; | 2.64; 63.9; 2.64; |
| | 0.0973 | 0.0543 | 0.0452 |
| G = nominal | 3.58; 67.1; 3.58; | 2.76; 61.5; 2.76; | 2.38; 60.1; 2.38; |
| | 0.0888 | 0.0530 | 0.0383 |

TABLE 5
Variation in long indices L-MIX, L-PIX, L-RIX, and L-IMIX,
respectively (see the text), for the yeast tissue model as a function
of acidity (pH) and glucose concentration G.

| | | |
|---|---|---|
| pH = 4.1 | pH = 5.4 | pH~7.3 |

Index variation normalized to percent per 100 mg/dl

| | | | |
|---|---|---|---|
| G = 800 mg/dl | 2.6; 0.9; 2.5; 5.7 | 3.7; 1.7; 3.7; 8.1 | 2.7; 1.4; 2.7; |
| | | | 7.1 |
| G = 400 mg/dl | 1.0; 0.5; 1.0; 2.4 | −0.1; 0.9; −0.1; 0.6 | 2.7; 1.6; 2.7; |
| | | | 4.5 |
| G = nominal | 0.0; 0.0; 0.0; 0.0 | 0.0; 0.0; 0.0; 0.0 | 0.0; 0.0; 0.0; |
| | | | 0.0 |

Index variation normalized to percent per unit of pH

| | | | |
|---|---|---|---|
| G = 800 mg/dl | 0.0; 0.0; 0.0; 0.0 | −15.9; −2.2; −15.7; | −15.3; −2.2; |
| | | −36.9 | −15.2; −35.7 |
| G = 400 mg/dl | 0.0; 0.0; 0.0; 0.0 | −27.4; −5.7; −27.4; | −12.9; −2.2; |
| | | −60.9 | −12.9; −36.0 |
| G = nominal | 0.0; 0.0; 0.0; 0.0 | −22.9; −7.0; −22.9; | −15.8; −3.6; |
| | | −52.0 | −15.8; −41.2 |

TABLE 6
Changes in absolute values of Z, φ, Re Z, and Im Z, respectively, for
the yeast tissue model (see the text), given in terms of percent per 100 mg/ml
glucose or percent per unit of acidity (pH), at different frequencies f. G is the
glucose concentration.

| | | |
|---|---|---|
| pH = 4.1 | pH = 5.4 | pH~7.3 |

At f = 20 kHz, in percent per 100 mg/dl

| | | | |
|---|---|---|---|
| G = 800 mg/dl | 4.1; 2.7; 4.1; 7.5 | 6.1; 3.4; 6.1; 11.0 | 6.1; 4.2; 6.1; 11.5 |
| G = 400 mg/dl | 1.1; 1.7 1.1; 2.5 | 2.2; 0.0; 2.2; 2.9 | 4.5; 2.8; 4.5; 6.3 |
| G = nominal | 0; 0.0; 0.0; 0.0 | 0.0; 0.0; 0.0; 0.0 | 0.0; 0.0; 0.0; 0.0 |

At f = 20 kHz, in percent per unit of pH

| | | | |
|---|---|---|---|
| G = 800 mg/dl | 0.0; 0.0; 0.0; 0.0 | −17.0; −16.5; −17.0; | −14.1; −13.0; −14.1; −34.0 |
| | | −38.5 | |
| G = 400 mg/dl | 0.0; 0.0; 0.0; 0.0 | −24.1; −28.0; −24.1;; | −13.8; −15.6; −13.8; −37.5 |
| | | −56.7 | |
| G = nominal | 0.0; 0.0; 0.0; 0.0 | −28.3 −21.0; −28.3; | −19.5; −17.4; −19.5; −46.9 |
| | | −58.8 | |

At f = 500 kHz, percent per 100 mg/dl

| | | | |
|---|---|---|---|
| G = 800 mg/dl | 3.4; 1.8; 2.8; 5.5 | 5.3; 3.1; 4.6; 9.5 | 5.5; 3.1; 4.9; 9.7 |
| G = 400 mg/dl | 0.9; 0.6; 0.7; 1.5 | 1.9; 1.2; 1.7; 3.1 | 4.1; 2.7; 3.7; 7.1 |
| G = nominal | 0.0; 0.0; 0.0; 0.0 | 0.0; 0.0; 0.0; 0.0 | 0.0; 0.0; 0.0; 0.0 |

At f = 500 kHz, percent per unit of pH

| | | | |
|---|---|---|---|
| G = 800 mg/dl | 0.0; 0.0; 0.0; 0.0 | −11.6; −14.7; −7.3; | −10.1; −12.0; −6.9; −24.5 |
| | | −26.8 | |
| G = 400 mg/dl | 0.0; 0.0; 0.0; 0.0 | −18.5; −20.9; −13.8; | −10.3; −12.4; −7.7; −25.7 |
| | | −42.7 | |
| G = nominal | 0.0; 0.0; 0.0; 0.0 | −22.3; −23.0; −17.3; | −15.5; −16.0; −12.3; −37.7 |
| | | −49.8 | |

At f = 5 MHz, percent per 100 mg/dl

| | | | |
|---|---|---|---|
| G = 800 mg/dl | 1.3; 0.9; −2.1; 1.7 | 1.8; 1.7; −3.2; 2.7 | 2.8; 1.5; −0.9; 3.7 |
| G = 400 mg/dl | 0.1; 0.5; −1.5; 0.3 | 2.3; 0.9; 0.2; 2.8 | 1.5; 1.6; −2.0; 2.4 |
| G = nominal | 0.0; 0.0; 0.0; 0.0 | 0.0; 0.0; 0.0; 0.0 | 0.0; 0.0; 0.0; 0.0 |

At f = 5 MHz, percent per unit of pH

| | | | |
|---|---|---|---|
| G = 800 mg/dl | 0.0; 0.0; 0.0; 0.0 | −1.0; −2.5; 8.7; −2.2 | 0.8; −2.4; 10.6; −0.2 |
| G = 400 mg/dl | 0.0; 0.0; 0.0; 0.0 | 2.5; −6.1; 21.0; −0.2 | −0.7; −2.4; 6.4; −1.9 |
| G = nominal | 0.0; 0.0; 0.0; 0.0 | −4.1; −7.2; 14.9; −7.8 | −2.5; −3.8; 7.1; −4.6 |

1.-44. (canceled)

45. A device for non-invasive determination of an estimate of glucose in the blood of a subject, comprising:
an electrically conducting probe comprising a plurality of electrodes adapted to measure the impedance of the tissue of a subject,
at least one sensing device adapted to sense at least one concentration acidity (pH) in the tissue of a subject,
wherein said electrically conducting probe and said sensing device are adapted to substantially simultaneously measure the impedance of the tissue and sense a concentration of acidity (pH) in the tissue, respectively, and
a blood glucose determining unit adapted to determine an estimate of blood glucose of said subject based on said tissue impedance and said acidity (pH) concentration.

46. A device for non-invasive determination of an estimate of blood glucose in the blood of a subject, comprising:
an electrically conducting probe comprising a plurality of electrodes adapted to measure a value of the impedance of the tissue of a subject,
wherein at least one of said plurality of electrodes is adapted for, when being in voltage mode, sense a signal representing a value of the concentration of acidity (pH) in the tissue of a subject, and
wherein said electrically conducting probe is adapted to substantially simultaneously obtain said value of the impedance of the tissue of a subject and said value of the concentration of acidity (pH) in the tissue of a subject, and
a blood glucose determining unit adapted to determine an estimate of blood glucose of said subject based on said tissue impedance and said acidity (pH) concentration.

47. The device according to claim 46, wherein at least one of said plurality of electrodes is made of an acidity-sensitive material.

48. The device according to claim 46 or 47, wherein said at least one of said plurality of electrodes is made of iridium, antimony, palladium, ruthenium, bismuth, zirkonium, oxides of iridium, antimony, palladium, ruthenium, bismuth, or zirkonium, or compositions of iridium, antimony, palladium, ruthenium, bismuth, or zirkonium, or oxides of iridium, antimony, palladium, ruthenium, bismuth, or zirkonium.

49. The device according to claim 45, wherein said electrically conducting probe comprises one pair of electrodes, wherein one electrode is a current injection electrode and the other is a voltage sensing electrode, or wherein said electrically conducting probe comprises two pairs of electrodes, each pair being a current injection electrode and a voltage sensing electrode.

50. The device according to claim 45 or 46, wherein said blood glucose determining unit is adapted to send an alert signal if said blood glucose estimate is below or above predetermined reference glucose levels.

51. The device according to claim 45 or 46, further comprising an insulin delivery device adapted to deliver an insulin dose based on said blood glucose estimate, and wherein said blood glucose determining unit is adapted to send a blood glucose estimate signal to said insulin delivery device to initiate said insulin dose delivery.

52. The device according to claim 45 or 46, wherein said device is adapted to be kept in continual contact with the tissue of said subject, and wherein said device optionally is adapted for periodic determination of said estimate of blood glucose.

53. The device according to claim 45, wherein said at least one sensing device comprises an ion-selective electrode, preferably a flat pH glass electrode or wherein said at least one sensing device comprises an acidity-sensitive field effect transistor.

54. The device according to claim 45 or 46, wherein said plurality of electrodes are arranged in a matrix or an array adapted for placement on a tissue surface of a subject, said device further comprising a processing unit, adapted for generating electrical impedance tomography images in the impedance domain, or wherein said plurality of electrodes and said at least one sensing device are arranged in a matrix or array adapted for placement on a tissue surface of a subject, said device further comprising a processing unit, adapted for generating electrical impedance tomography images in the impedance and ion concentration domain, to be related to the tissue structure underlying the tissue surface of a the subject, wherein said processing unit preferably is integrated with said blood glucose determining unit.

55. A method for non-invasive determination of an estimate of blood glucose in the blood of a subject, said method comprising the steps of:
(a) placing an electrically conducting probe against a tissue surface of a subject wherein said probe comprises a plurality of electrodes and at least one sensing device or sensor adapted for sensing a concentration of acidity (pH) in the tissue;
(b) passing an electrical current through the electrodes to obtain a value of the impedance of the tissue;
(c) using said at least one sensing device or sensor to obtain at least one value of a concentration of acidity (pH) in the tissue,
(d) wherein said value of the impedance of the tissue and said at least one value of a concentration of acidity (pH) in the tissue are obtained substantially simultaneously; and
(e) determining an estimate of blood glucose in blood of a subject on the basis of said at least one value of a concentration of acidity (pH) and said impedance value.

56. A method for non-invasive determination of an estimate of blood glucose in blood of a subject, said method comprising the steps of:
(a) placing an electrically conducting probe against a tissue surface of a subject, wherein said probe comprises a plurality of electrodes, and wherein at least one of said plurality of electrodes is adapted for, when being in voltage mode, sensing a signal representing a value of a concentration of acidity (pH) in the tissue of a subject;
(b) passing an electrical current through the electrodes to obtain a value of the impedance of the tissue;
(c) using said at least one electrode adapted for sensing a signal representing a value of a concentration of acidity (pH) in the tissue of a subject to obtain at least one value of a concentration of acidity (pH) in the tissue,
(d) wherein said value of the impedance of the tissue and said at least one value of a concentration of acidity (pH) in the tissue are obtained substantially simultaneously; and
(e) determining an estimate of blood glucose in blood of a subject on the basis of said at least one value of a concentration of acidity (pH) and said impedance value.
